(19) United States
(12) Patent Application Publication
(10) Pub. No.: US 2020/0283836 A1
(43) Pub. Date: Sep. 10, 2020

(54) TELOMERE-CONTROLLING GENE FAMILY DISCOVERED IN MOUSE THYMUS LYMPHOMA CELL IRRADIATED WITH LOW-DOSE RATE LOW-LEVEL RADIATION, AND METHOD FOR DETECTING SAME

(71) Applicant: KOREA HYDRO & NUCLEAR POWER CO., LTD., Gyeongsangbuk-do (KR)

(72) Inventors: Hee Sun KIM, Gyeonggi-do (KR); Hoon CHOI, Seoul (KR); Yun-Mi BAEK, Seoul (KR); Byulnim HWANG, Gyeongsangbuk-do (KR); Hee Youn SHIM, Seoul (KR)

(21) Appl. No.: 16/649,281

(22) PCT Filed: Oct. 25, 2017
(86) PCT No.: PCT/KR2017/011839

§ 371 Date: Mar. 20, 2020

(30) Foreign Application Priority Data
Sep. 26, 2017 (KR) 10-2017-0124184

(51) Int. Cl. C12Q 1/6851; C12Q 1/6886; C12N 13/00

(52) U.S. Cl. CPC C12Q 1/6851; C12N 13/00; C12Q 1/6886

(57) ABSTRACT

The present invention relates to a method of detecting a telomere-controlling gene family that responds to low-dose-rate low-level radiation, and more particularly to a method of detecting a telomere-controlling gene that is expressed in common or alone by applying low-dose low-level radiation to mouse thymic lymphoma cells and then measuring the expression levels of telomere-controlling genes. By providing the method of detecting the telomere-controlling gene family that responds to low-dose-rate low-level radiation according to the present invention, telomere-controlling genes can be used as low-level-radiation telomere-controlling genes with which the relationship between radiation exposure and carcinogenesis of industrial and medical workers can be evaluated, as low-level-radiation telomere-controlling genes with which cancer progression and the extent of treatment of cancer patients can be evaluated, and also as a low-level telomere-controlling gene recovery indicator with which the causal relationship between radiation and carcinogenesis can be evaluated.

### TECHNICAL FIELD

The present invention relates to a method of detecting a telomere-controlling gene family that responds to low-dose-rate low-level radiation, and more particularly to a method of detecting a telomere-controlling gene that is expressed in common or alone by applying low-dose low-level radiation to mouse thymic lymphoma cells and then measuring the expression levels of telomere-controlling genes.

### BACKGROUND ART

In order to determine the scientific mechanism of the effect of radiation on humans, which is currently a social issue, many attempts are being made to obtain scientific evidence at the cellular and molecular level as well as the individual level, but are still inadequate because there have been no reproducible results for the low-dose radiation range of 100 mSv or less.

Radiation damages hematopoietic and intestinal canal tissues, causing leucopenia, and increases the permeation of normal flora from the intestinal mucosa, and is therefore known to be a factor mainly associated with diseases such as cancer by impairing specific or nonspecific immune defense mechanisms to thus reduce resistance to infectious diseases. These radiation effects include damage to the blood tissue barrier, a decrease in the number of phagocytes, decreased ability to kill fed organisms, a decrease in serum complement levels and impaired immune responses.

When exposed to radiation of 2 to 7 Gy, the number of lymphocytes is significantly reduced, reaching a minimum within a few hours and then gradually increasing over time, but it may take three to four weeks after exposure to radiation to return to normal levels. On the other hand, low-dose radiation of about 0.25 to 1 Gy delays the formation of antibodies compared to animals that have not been irradiated, and very high antibody peak titers are temporarily observed. However, the mechanisms of low-dose radiation effects in the range of 0.25 Gy or less are not clear, and remain disputed to date in opposition to the hormesis theory [Stebbing, 1982].

Real-world application of low-dose radiation has been reported to promote individual growth and increase immune function and lifespan [Luckey T. D. et al., 1982]. There is also a research report on a hermetic response to radiation in humans, and Bloom et al. (1987) reported that cellular immunity in humans is enhanced at 0.5 Gy or less. Nambi and Soman (1987) reported reduced cancer incidence at 0.03 μSv (0.3 mrem) per year. However, when studying the impact of radiation exposure on humans, it takes a long time before the effect of low-dose radiation of 100 mSv or less is exhibited in humans and there are many limitations in detecting the same. This research is only for observing phenomenological effects, and it still remains controversial because no specific mechanism of influence is known.

“Telomere”, which is a portmanteau of Greek “telos (end)” and “meros (part)”, refers to the terminus of a chromosome in which six nucleotides (AATCCC, TTAGGG, etc.) are repetitively arranged thousands of times. That is, a telomere is the nucleotide sequence moiety of the end of a chromosome. In this moiety, as cell division progresses, it is found that the length becomes shorter and consequently only knots are left, and cell replication stops, thus resulting in death, which is presumed to be the determinant of aging and lifespan. Contrary to the shortening of telomere through division in normal cells, it is confirmed that the telomere is no longer shortened and maintained unchanged in cancer cells, which plays an important role in the maintenance and development of cancer. Hence, although controlling the telomere length is thought to be an important target for cancer treatment, it is not known how low-dose-rate low-level radiation affects telomere length.

In the present invention, how genes known to be involved in telomere length regulation respond to low-dose-rate low-level radiation at the RNA and protein levels is confirmed. The genes discovered to respond sensitively to low-dose-rate low-level radiation in the present invention belong to a gene family already known to play an important role in telomere length regulation. Moreover, the genes identified in the present invention are not known as genes sensitive to low-dose-rate low-level radiation, and in particular, by discovering genes that respond differently to low-dose-rate low-level radiation at the RNA and protein levels, low-dose-rate low-level radiation shows that it is possible to regulate protein expression independent of RNA expression, rather than regulating protein expression through control of RNA expression. Ultimately, the present inventors have developed and ascertained a method of detecting the telomere-controlling gene family of cells responding differently to low-dose low-level radiation stimulation in the range of 0.25 Gy or less, at the RNA and protein levels, thus culminating in the present invention.

### DISCLOSURE

### Technical Problem

In the study of radiation-assisted cancer therapy by the present inventors, study results obtained using mice may be generally applicable directly to humans or animals, but there is a limitation in that such results are difficult to interpret at the molecular level. In order to compensate for these drawbacks, molecular changes using mouse thymic lymphoma cells are measured, thus culminating in the present invention.

1) After application of low-dose-rate low-level radiation, high-dose-rate low-level radiation and high-level radiation to mouse thymic lymphoma cells, changes in telomere-controlling genes are observed, 2) among them, telomere-controlling genes that respond sensitively to low-dose-rate low-level radiation are verified and functions thereof are analyzed, and 3) the effectiveness of low-dose-rate low-level radiation is confirmed in the treatment of cancer targeted to telomere length regulation.

Accordingly, an objective of the present invention is to provide a method of detecting a telomere-controlling gene family that responds to low-dose-rate low-level radiation, including (1) irradiating mouse thymic lymphoma cells with low-dose-rate low-level radiation, high-dose-rate low-level radiation and high-level radiation, (2) observing changes in genes already known as telomere-controlling genes in the irradiated cells, (3) detecting telomere-controlling genes that are expressed alone by the low-dose-rate low-level radiation, among the telomere-controlling genes changes of which are observed, and (4) amplifying the detected genes and measuring the expression level thereof.

### Technical Solution

In order to accomplish the above objective, the present invention provides a method of detecting a telomere-controlling gene that responds to low-dose-rate low-level radiation, including (1) irradiating lymphoma cells with low-dose-rate low-level radiation and high-dose-rate low-level radiation, (2) observing changes in genes already known as telomere-controlling genes in the cells irradiated in step (1), (3) detecting telomere-controlling genes that are expressed alone by the low-dose-rate low-level radiation, among the telomere-controlling genes changes of which are observed in step (2), and (4) amplifying the genes detected in step (3) and measuring an expression level thereof.

In step (1), the low-dose-rate low-level radiation may be 23.22 mGy/hr.

In step (1), the cells may be cultured such that a total cumulative dose of the low-dose-rate low-level radiation of 23.22 mGy/hr is 557.28 mGy.

In step (4), the gene involved in telomere may be amplified using a primer selected from among tert, wrap53, dkc1, ctc1, pot1, tpp1, tin2, trf1, trf2, rap1, rtel1 and telo2 gene sequences.

In step (4), the expression level may be measured through a quantitative nucleic acid amplification process and a special protein detection test.

### Advantageous Effects

The present invention provides a method of detecting a telomere-controlling gene family that responds to low-dose-rate low-level radiation.

Accordingly, telomere-controlling genes can be utilized as low-level-radiation telomere-controlling genes with which the relationship between radiation exposure and carcinogenesis of industrial and medical workers can be evaluated, as low-level-radiation telomere-controlling genes with which cancer progression and the extent of treatment of cancer patients can be evaluated, and also as a low-level telomere-controlling gene recovery indicator with which the causal relationship between radiation and carcinogenesis can be evaluated.

### DESCRIPTION OF DRAWINGS

FIG. 1 is graphs showing the results of measurement of relative expression levels using a nucleic acid amplification process for changes in the expression of telomere length regulation cancer suppression indicator genes depending on the dose rate and dose of Experimental Example 2;

FIG. 2 is a graph showing the results of analysis of changes in cancer-suppression-related telomere-controlling protein depending on the dose rate and dose of Experimental Example 3, in which the protein expression levels of six genes (TRF2, Tin2, Rap1, Pot1, Rtel1, CTC1) are changed; and

FIG. 3 schematically shows changes in telomere-controlling genes induced by low-dose-rate low-level radiation in mouse thymic lymphoma cells, in which TRF2, Tin2, Rap1 and Pot1 increase the expression of genes and Rtel1 and CTC1 decrease the expression of genes, which reduces the telomere length and lowers the possibility of carcinogenesis.

### BEST MODE

Hereinafter, a detailed description will be given of the present invention.

An aspect of the present invention pertains to a method of detecting a telomere-controlling gene that responds to low-dose-rate low-level radiation, including (1) irradiating lymphoma cells with low-dose-rate low-level radiation and high-dose-rate low-level radiation, (2) observing changes in genes already known as telomere-controlling genes in the cells irradiated in step (1), (3) detecting telomere-controlling genes that are expressed alone by the low-dose-rate low-level radiation, among the telomere-controlling genes changes of which are observed in step (2), and (4) amplifying the genes detected in step (3) and measuring the expression level thereof. Here, the lymphoma cells may be mouse thymic lymphoma cells (EL4), and moreover, in step (1), cells irradiated with high-level radiation may be added as a comparative group.

In step (1), the low-dose-rate low-level radiation is 23.22 mGy/hr.

In step (1), the total cumulative dose of the low-dose-rate low-level radiation of 23.22 mGy/hr is 557.28 mGy.

In step (4), the gene involved in telomere is amplified using a primer selected from among tert, wrap53, dkc1, ctc1, pot1, tpp1, tin2, trf1, trf2, rap1, rtel1 and telo2 gene sequences.

In step (4), the expression level is measured through a quantitative nucleic acid amplification process and a special protein detection test.

Based on changes in the telomere-controlling genes attributable to the low-dose-rate low-level radiation in the mouse thymic lymphoma cells, TRF2, Tin2, Rap1 and Pot1 were found to increase the expression of genes and Rtel1 and CTC1 were found to decrease the expression of genes, indicating that six genes are changed.

A better understanding of the present invention will be given through the following examples. These examples are merely set forth to illustrate the present invention, but are not to be construed as limiting the scope of the present invention, as will be apparent to those skilled in the art.

Six genes (TRF2, Tin2, Rap1, Pot1, Rtel1, CTC1) responding sensitively to low-dose-rate low-level radiation discovered in the present invention have not been claimed before, but there has been a report on the characteristics thereof associated with telomere length regulation. TRF2 is known to induce rapid degradation of telomeres independent of telomerase (Munoa et al., 2006). Tin2 is known to inhibit telomere extension by telomerase (Kim et al., 1999). The lack of Rap1 extends the telomere length, suggesting that Rap1 has a function of suppressing the telomere length (O'Connor et al., 2004). Pot1 has been reported to inhibit telomere length extension (Kendellen et al., 2009). Rtel1 is known to play an important role in maintaining the telomere length (Uringa et al., 2010). CTC1 is known to inhibit shortening of the telomere length and apoptosis in human melanoma cells (Luo et al., 2014).

It is known that regulating the telomere length may be an important treatment method for cancer suppression, but a lot of research is still required on how to inhibit maintenance of telomere length in cancer cells upon real-world application. The present invention investigates how the expression of telomere length regulation genes in cancer cells is changed by low-dose-rate low-level radiation and analyzes whether cancer may be suppressed therethrough.

### Experimental Example 1. Preparation of Mouse Thymic Lymphoma Cells and Irradiation

The mouse thymic lymphoma cells were purchased from ATCC and stabilized in a 5% CO₂ incubator at 37° C. The mouse thymic lymphoma cells were irradiated with low-level radiation at a cumulative dose of 557.28 mGy, and were divided into two groups in order to apply low-dose-rate low-level radiation and high-dose-rate low-level radiation respectively thereto. Cells irradiated with high-level radiation of 2 Gy were also prepared for comparison with the effect of low-level radiation.

### Experimental Example 2. Measurement of Expression Levels of Telomere-Controlling Genes and Primers

Telomere-controlling genes responding sensitively to low-dose-rate low-level radiation in the mouse thymic lymphoma cells were discovered, and the expression levels necessary to interpret the functions thereof were measured using a quantitative nucleic acid amplification process and a special protein detection test.

Meanwhile, the primer sequences used to measure the expression levels of the sensitively responding telomere-controlling genes in the mouse thymic lymphoma cells irradiated with low-dose-rate low-level radiation (23.22 mGy/hr) are shown in Table 1 below.

TABLE 1
Primer sequences necessary to measure expression
levels of telomere length regulation genes in
mouse thymic lymphoma cells irradiated with low-
dose-rate low-level radiation (23.22 mGy/hr)

| Gene No. | Gene | Forward (5′→3′) | Reverse (5′→3′) |
|---|---|---|---|
| NM_009354 | tea | CAAGGCCAAGTCCAC | CACTGGCATCTGAAT |
| | | AAGTC | CCTGC |
| NM_144824 | wrap53 | GTCGGAGGAGCGACT | GCTGGGGTTGGTCAT |
| | | CTTAG | CACC |
| NM_001030307 | dkc1 | AAAGACCGGAAGCCA | GCCACTGAGAAGTGT |
| | | TTACAAG | CTAATTGA |
| NR_001579 | ctc1 | TCCGACCCGTTAAGC | TTCACTCTGGTCAGC |
| | | TTTCT | AGAGG |
| NM_133931 | pot1 | TTGGTTTCAACAGCT | GGAGGGCTTCATAGT |
| | | CCCTATAC | TTCCACT |
| NM_009906 | tpp1 | GAGTCTCACTTTTGC | CTCCAGGGTTAGGTA |
| | | GCTGAA | CTTTCCA |
| NM_145705 | tin2 | TCTAAGTTGGAGTCA | AATGTCCACCCCATG |
| | | GCCGG | TCCAT |
| NM_009352 | trf1 | CAGCAGTCTACAGAA | ACTGAAATCTGATGG |
| | | ACTGAACC | AGCACG |
| NM_001083118 | trf2 | TCTGTCGCGCATTGA | ATTCCAAGGGTGTGA |
| | | AGAAG | GCTCA |
| NM_020584 | rap1 | TGCCTTGTGGAAAGC | TGTTCTGTGGCTCTC |
| | | GATG | CGCTAT |
| NM_001001882 | rtel1 | CTTTGGCCATGTCAT | AGAGAGGGAGTAGCT |
| | | CCGAG | GGACA |
| NM_001163661 | telo2 | CTCTGGTGACCTTCG | GCTAAGGTGTGGGTC |
| | | ACCTT | AGTCT |

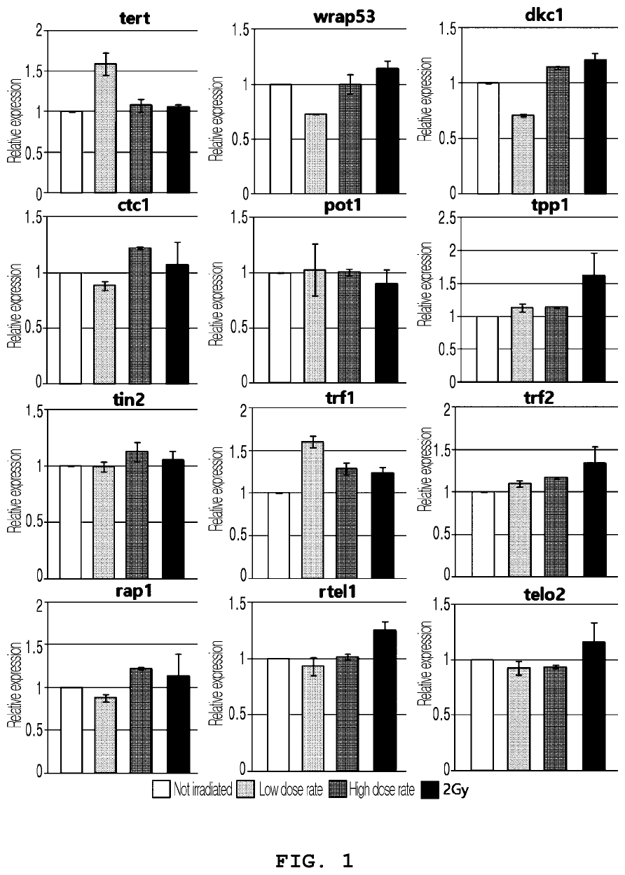

In order to measure the expression levels of 12 genes (Table 1) known to be involved in telomere length regulation in the mouse thymic lymphoma cells irradiated with low-dose-rate low-level radiation (23.22 mGy/hr), a nucleic acid amplification process was used. As shown in FIG. 1, in the mouse thymic lymphoma cells irradiated with low-dose-rate low-level radiation, five genes including tert, wrap53, dkc1, ctc1 and trf1 responded specifically and sensitively to radiation, and thus the expression levels thereof were increased or decreased.

### Experimental Example 3. Protein Analysis of Mouse Thymic Lymphoma Cells Irradiated with Low-Dose-Rate Low-Level Radiation

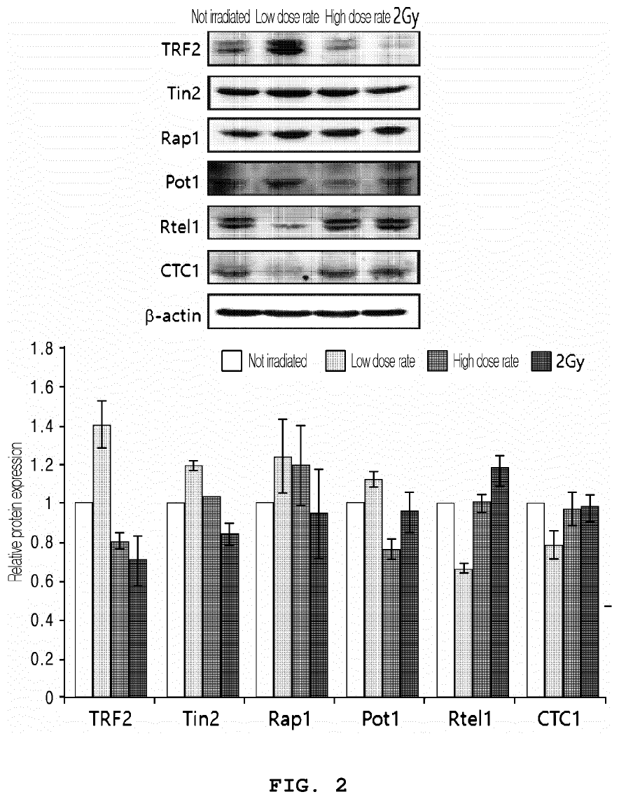

Proteins irradiated with low-dose-rate low-level radiation (23.22 mGy/hr) and with high-dose-rate low-level radiation (800 mGy/min, total dose: 557.28 mGy) as a control were analyzed. As shown in FIG. 2, in the mouse thymic lymphoma cells irradiated with low-dose-rate low-level radiation, TRF2, Tin2, Rap1, Pot1, Rtel1 and CTC1 proteins responded specifically and sensitively, and thus the expression levels thereof were increased or decreased.

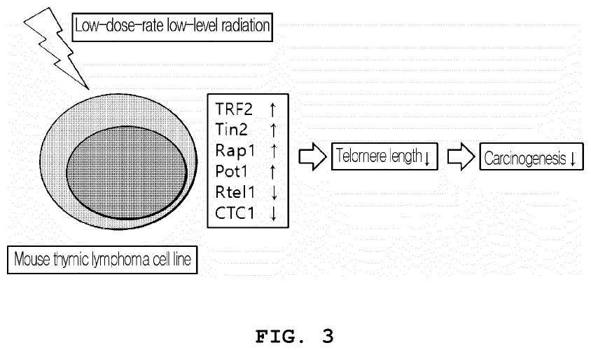

Based on the above results, six genes (TRF2, Tin2, Rap1, Pot1, Rtel1, CTC1) were discovered as the telomere length regulation genes responding sensitively to the low-dose-rate low-level radiation, and changes in RNA and protein levels due thereto were evaluated. As summarized in FIG. 3, changes in the telomere-controlling genes by the low-dose-rate low-level radiation in the mouse thymic lymphoma cells are shown. TRF2, Tin2, Rap1 and Pot1 increased the expression of genes and Rtel1 and CTC1 decreased the expression of genes, thereby reducing the telomere length and lowering the possibility of carcinogenesis.

Although specific embodiments of the present invention have been disclosed in detail as described above, it will be obvious to those skilled in the art that such description is merely of preferable exemplary embodiments and is not to be construed to limit the scope of the present invention. Therefore, the substantial scope of the present invention will be defined by the appended claims and equivalents thereof.

### SEQUENCE LISTING FREE TEXT

Electronic file attached

1. A method of detecting a telomere-controlling gene that responds to low-dose-rate low-level radiation, comprising the steps of:
irradiating lymphoma cells with low-dose-rate low-level radiation and high-dose-rate low-level radiation;
observing changes in genes already known as telomere-controlling genes in the irradiated lymphoma cells;
detecting telomere-controlling genes that are expressed alone by the low-dose-rate low-level radiation, among the observed changes in telomere-controlling genes; and
amplifying the detected telomere-controlling genes and measuring an expression level thereof.

2. The method of claim 1, wherein:
in the irradiating step, the low-dose-rate low-level radiation is 23.22 mGy/hr.

3. The method of claim 1, wherein:
in the irradiating step, the cells are cultured such that a total cumulative dose of the low-dose-rate low-level radiation of 23.22 mGy/hr is 557.28 mGy.

4. The method of claim 1, wherein:
in the amplifying step, the gene involved in telomere is amplified using a primer selected from the group consisting of: tert, wrap53, dkc1, ctc1, pot1, tpp1, tin2, trf1, trf2, rap1, rtel1 and telo2 gene sequences.

5. The method of claim 1, wherein:
in the amplifying step, the expression level is measured through a quantitative nucleic acid amplification process and a special protein detection test.


SEQUENCE LISTING (24 sequences)

SEQ ID NO: 1 - DNA, 20 bp, Artificial Sequence
  tert(NM_009354) Forward Primer
caaggccaagtccacaagtc

SEQ ID NO: 2 - DNA, 20 bp, Artificial Sequence
  tert(NM_009354) Backward Primer
cactggcatctgaatcctgc

SEQ ID NO: 3 - DNA, 20 bp, Artificial Sequence
  wrap53(NM_144824) Forward Primer
gtcggaggagcgactcttag

SEQ ID NO: 4 - DNA, 19 bp, Artificial Sequence
  wrap53(NM_144824) Backward Primer
gctggggttggtcatcacc

SEQ ID NO: 5 - DNA, 22 bp, Artificial Sequence
  dkc1(NM_001030307) Forward Primer
aaagaccggaagccattacaag

SEQ ID NO: 6 - DNA, 23 bp, Artificial Sequence
  dkc1(NM_001030307) Backward Primer
gccactgagaagtgtctaattga

SEQ ID NO: 7 - DNA, 20 bp, Artificial Sequence
  ctc1(NR_001579) Forward Primer
tccgacccgttaagctttct

SEQ ID NO: 8 - DNA, 20 bp, Artificial Sequence
  ctc1(NR_001579) Backward Primer
ttcactctggtcagcagagg

SEQ ID NO: 9 - DNA, 23 bp, Artificial Sequence
  pot1(NM_133931) Forward Primer
ttggtttcaacagctccctatac

SEQ ID NO: 10 - DNA, 22 bp, Artificial Sequence
  pot1(NM_133931) Backward Primer
ggagggcttcatagtttccact

SEQ ID NO: 11 - DNA, 21 bp, Artificial Sequence
  tpp1(NM_009906) Forward Primer
gagtctcacttttgcgctgaa

SEQ ID NO: 12 - DNA, 22 bp, Artificial Sequence
  tpp1(NM_009906) Backward Primer
ctccagggttaggtactttcca

SEQ ID NO: 13 - DNA, 20 bp, Artificial Sequence
  tin2(NM_145705) Forward Primer
tctaagttggagtcagccgg

SEQ ID NO: 14 - DNA, 20 bp, Artificial Sequence
  tin2(NM_145705) Backward Primer
aatgtccaccccatgtccat

SEQ ID NO: 15 - DNA, 23 bp, Artificial Sequence
  trf1(NM_009352) Forward Primer
cagcagtctacagaaactgaacc

SEQ ID NO: 16 - DNA, 21 bp, Artificial Sequence
  trf1(NM_009352) Backward Primer
actgaaatctgatggagcacg

SEQ ID NO: 17 - DNA, 20 bp, Artificial Sequence
  trf2(NM_001083118) Forward Primer
tctgtcgcgcattgaagaag

SEQ ID NO: 18 - DNA, 20 bp, Artificial Sequence
  trf2(NM_001083118) Backward Primer
attccaagggtgtgagctca

SEQ ID NO: 19 - DNA, 19 bp, Artificial Sequence
  rap1(NM_020584) Forward Primer
tgccttgtggaaagcgatg

SEQ ID NO: 20 - DNA, 21 bp, Artificial Sequence
  rap1(NM_020584) Backward Primer
tgttctgtggctctccgctat

SEQ ID NO: 21 - DNA, 20 bp, Artificial Sequence
  rtel1(NM_001001882) Forward Primer
ctttggccatgtcatccgag

SEQ ID NO: 22 - DNA, 20 bp, Artificial Sequence
  rtel1(NM_001001882) Backward Primer
agagagggagtagctggaca

SEQ ID NO: 23 - DNA, 20 bp, Artificial Sequence
  telo2(NM_001163661) Forward Primer
ctctggtgaccttcgacctt

SEQ ID NO: 24 - DNA, 20 bp, Artificial Sequence
  telo2(NM_001163661) Backward Primer
gctaaggtgtgggtcagtct